(19) United States
(12) Patent Application Publication
(10) Pub. No.: US 2024/0131040 A1
(43) Pub. Date: Apr. 25, 2024

(54) COMPOSITIONS AND METHODS RELATED TO CANNABINOID ANIONS

(71) Applicant: NATURAL EXTRACTION SYSTEMS, LLC, Boulder, CO (US)

(72) Inventors: C. Russell THOMAS, Boulder, CO (US); Douglas G. METCALF, Boulder, CO (US)

(73) Assignee: NATURAL EXTRACTION SYSTEMS, LLC, Boulder, CO (US)

(21) Appl. No.: 18/546,084

(22) PCT Filed: Feb. 10, 2022
(86) PCT No.: PCT/US2022/015914

§ 371 Date: Aug. 10, 2023

Related U.S. Application Data
(60) Provisional application No. 63/148,047, filed on Feb. 10, 2021.
(60) Provisional application No. 63/154,480, filed on Feb. 26, 2021.
(60) Provisional application No. 63/154,507, filed on Feb. 26, 2021.
(60) Provisional application No. 63/154,538, filed on Feb. 26, 2021.
(60) Provisional application No. 63/154,579, filed on Feb. 26, 2021.
(60) Provisional application No. 63/191,814, filed on May 21, 2021.
(60) Provisional application No. 63/191,830, filed on May 21, 2021.
(60) Provisional application No. 63/191,844, filed on May 21, 2021.
(60) Provisional application No. 63/191,872, filed on May 21, 2021.
(60) Provisional application No. 63/194,810, filed on May 28, 2021.
(60) Provisional application No. 63/228,563, filed on Aug. 2, 2021.
(60) Provisional application No. 63/228,572, filed on Aug. 2, 2021.
(60) Provisional application No. 63/228,594, filed on Aug. 2, 2021.
(60) Provisional application No. 63/254,429, filed on Oct. 11, 2021.
(60) Provisional application No. 63/256,452, filed on Oct. 15, 2021.
(60) Provisional application No. 63/282,123, filed on Nov. 22, 2021.

(51) Int. Cl. A61K 31/00; A61K 9/00; A61P 25/02; A61P 25/04; A61P 25/08; A61P 25/22; A61P 25/28; A61P 29/00

(52) U.S. Cl. CPC A61K 31/658; A61K 9/0053; A61P 25/02; A61P 25/04; A61P 25/08; A61P 25/22; A61P 25/28; A61P 29/00

(57) ABSTRACT

Various aspects of this disclosure relate to a composition, comprising a liquid phase that comprises a plurality of cations, an anion, a molecule, a base, and a solvent, wherein the plurality of cations, the anion, the molecule, and the base are solutes that are dissolved in the solvent; each cation of the plurality of cations is a metal cation or an ammonium cation; the molecule is a cannabinoid; the solvent is not water; the solvent has a solubility in water at 37 degrees Celsius of at least 25 grams per liter; each cation of the plurality of cations has a net charge of 1, 2, or 3; the anion has a net charge of −1; the molecule lacks a net charge; the base has a net charge of −1; the composition has a molar concentration for the anion, the molecule, the base, and each cation of the plurality of cations; the composition has an equivalent concentration for each cation of the plurality of cations, which is equal to the molar concentration of a cation multiplied by the net charge of the same cation; the composition has a combined equivalent concentration of the plurality of cations, which is equal to the sum of the equivalent concentrations of the cations of the plurality of cations; the combined equivalent concentration is no less than the molar concentration of the anion; the combined equivalent concentration is greater than the molar concentration of the base; the molar concentration of the anion is greater than the molar concentration of the molecule; the molar concentration of the solvent is at least 100 times greater than the molar concentration of the base; the liquid phase comprises the solvent at a greater concentration by mass than any other chemical species that is present in the liquid phase; the molecule has an acid dissociation constant in water of at least 50 femtomolar and no greater than 50 nanomolar for conversion of the molecule into the anion; the solvent has an acid dissociation constant in water of at least 50 attomolar for conversion of the solvent into the base; the acid dissociation constant of the molecule is at least 10 times greater than the acid dissociation constant of the solvent; the molecule has an octanol-water partition coefficient at 37 degrees Celsius that is greater than 1; the molecule has a solubility in water, which is the maximum thermodynamically-stable molar concentration of the molecule that can be dissolved in distilled water as a solute that is solvated by water at atmospheric pressure and 21 degrees Celsius; and the molar concentration of the anion is greater than the solubility of the molecule in water. In some embodiments, the composition is effective to inhibit TNF-alpha mediated signaling pathways in a human.

### CROSS-REFERENCE TO RELATED APPLICATIONS

This patent application claims priority to U.S. Provisional Patent Application No. 63/148,047, filed Feb. 10, 2021; U.S. Provisional Patent Application No. 63/154,480, filed Feb. 26, 2021; U.S. Provisional Patent Application No. 63/154,507, filed Feb. 26, 2021; U.S. Provisional Patent Application No. 63/154,538, filed Feb. 26, 2021; U.S. Provisional Patent Application No. 63/154,579, filed Feb. 26, 2021; U.S. Provisional Patent Application No. 63/191,814, filed May 21, 2021; U.S. Provisional Patent Application No. 63/191,830, filed May 21, 2021; U.S. Provisional Patent Application No. 63/191,844, filed May 21, 2021; U.S. Provisional Patent Application No. 63/191,872, filed May 21, 2021; U.S. Provisional Patent Application No. 63/194,810, filed May 28, 2021; U.S. Provisional Patent Application No. 63/228,563, filed Aug. 2, 2021; U.S. Provisional Patent Application No. 63/228,572, filed Aug. 2, 2021; U.S. Provisional Patent Application No. 63/228,594, filed Aug. 2, 2021; U.S. Provisional Patent Application No. 63/254,429, filed Oct. 11, 2021; U.S. Provisional Patent Application No. 63/256,452, filed Oct. 15, 2021; and U.S. Provisional Patent Application No. 63/282,123, filed Nov. 22, 2021, each of which is incorporated by reference in its entirety.

### BACKGROUND

Methods to improve the solubility, bioavailability, and pharmacokinetics of cannabinoids are desirable.

### SUMMARY

Cannabinoids are generally insoluble in water and glycerol. Some aspects of the disclosure relate to the discovery that the conversion of cannabinoid molecules into anions under alkaline conditions improves their solubility, bioavailability, and pharmacokinetics. Some aspects of the disclosure relate to the discovery that cannabigerol and related cannabinoids can inhibit tumor necrosis factor alpha (“TNF-alpha”) signaling pathways in animal models and that the cannabigerol anion can treat health conditions in humans that are sensitive to TNF-alpha signaling pathway inhibitors including Crohn's disease and plaque psoriasis. Some aspects of the disclosure relate to the discovery that cannabinoids can reduce pain and inflammation in both animal models and humans. Some aspects of the disclosure relate to the discovery that the topical administration of the cannabigerol anion can treat health conditions including peripheral neuropathy and plaque psoriasis in humans. Some aspects of the disclosure relate to the discovery that the oral administration of the cannabidiol anion can arrest active seizures in animals.

### DETAILED DESCRIPTION

Various aspects of this disclosure relate to a composition, comprising a liquid phase that comprises a plurality of cations, an anion, a molecule, a base, and a solvent, wherein the plurality of cations, the anion, the molecule, and the base are solutes that are dissolved in the solvent; each cation of the plurality of cations is a metal cation or an ammonium cation; the molecule is a cannabinoid; the solvent is not water; the solvent has a solubility in water at 37 degrees Celsius of at least 25 grams per liter; each cation of the plurality of cations has a net charge of 1, 2, or 3; the anion has a net charge of −1; the molecule lacks a net charge; the base has a net charge of −1; the composition has a molar concentration for the anion, the molecule, the base, and each cation of the plurality of cations; the composition has an equivalent concentration for each cation of the plurality of cations, which is equal to the molar concentration of a cation multiplied by the net charge of the same cation; the composition has a combined equivalent concentration of the plurality of cations, which is equal to the sum of the equivalent concentrations of the cations of the plurality of cations;

the combined equivalent concentration is no less than the molar concentration of the anion; the combined equivalent concentration is greater than the molar concentration of the base; the molar concentration of the anion is greater than the molar concentration of the molecule; the molar concentration of the solvent is at least 100 times greater than the molar concentration of the base; the liquid phase comprises the solvent at a greater concentration by mass than any other chemical species that is present in the liquid phase; the molecule has an acid dissociation constant in water of at least 50 femtomolar and no greater than 50 nanomolar for conversion of the molecule into the anion; the solvent has an acid dissociation constant in water of at least 50 attomolar for conversion of the solvent into the base; the acid dissociation constant of the molecule is at least 10 times greater than the acid dissociation constant of the solvent; the molecule has an octanol-water partition coefficient at 37 degrees Celsius that is greater than 1; the molecule has a solubility in water, which is the maximum thermodynamically-stable molar concentration of the molecule that can be dissolved in distilled water as a solute that is solvated by water at atmospheric pressure and 21 degrees Celsius; and the molar concentration of the anion is greater than the solubility of the molecule in water.

“Comprise” and “comprising” refer to open sets; a composition comprising a plurality of cations, for example, can also comprise one or more cations that are not included in the plurality of cations. “Ammonium cation” refers to both ammonium and aminium cations.

“Cannabinoid” refers to a cannabinoid chemical species that lacks a net charge, and “anion” refers to a cannabinoid chemical species that comprises a net negative charge. Various cannabinoid molecules and anions are described in PCT Patent Application Publication No. WO2020123809, PCT Patent Application Publication No. WO2020123976, PCT Patent Application Publication No. WO2020264199, U.S. Pat. Nos. 10,555,914, and 10,609,944, which are incorporated by reference for the chemical species that they disclose.

“Molar concentration” refers to concentrations in both liquid phases and solid phases, and when the molar concentration refers to a liquid phase, then the molar concentration is limited to the concentration of either a solvent or a solute that is dissolved in a solvent.

“Dissolved” refers to a solute that is solvated in a liquid phase; a chemical species that is present within a phase that is dispersed within a liquid phase, such as the dispersed phase of an emulsion, is not dissolved in the liquid phase; a chemical species that is non-covalently bound to any chemical species that is a solid in the absence of a solvent, such as a cyclodextrin, is not dissolved in a liquid phase.

“Distilled water” refers to water that lacks solutes other than dissolved gasses, and for the purposes of this disclosure, distilled water has a pH of 6.0 due to the presence of dissolved carbon dioxide.

In some embodiments, the liquid phase comprises water and hydroxide at a molar ratio of at least 100:1 and no greater than 10,000,000:1. In some specific embodiments, the liquid phase comprises water and hydroxide at a molar ratio of at least 100:1 and no greater than 100,000:1.

In some embodiments, the liquid phase comprises ethanol and ethoxide at a molar ratio of at least 100:1 and no greater than 10,000,000:1. In some specific embodiments, the liquid phase comprises ethanol and ethoxide at a molar ratio of at least 100:1 and no greater than 100,000:1.

In some embodiments, the liquid phase comprises propylene glycol and one or both of 2-hydroxypropane-1-oxide and 1-hydroxypropane-2-oxide at a molar ratio of at least 100:1 and no greater than 10,000,000:1 (propylene glycol:2-hydroxypropane-1-oxide and 1-hydroxypropane-2-oxide). In some specific embodiments, the liquid phase comprises propylene glycol and one or both of 2-hydroxypropane-1-oxide and 1-hydroxypropane-2-oxide at a molar ratio of at least 100:1 and no greater than 100,000:1.

In some embodiments, the liquid phase comprises glycerol and one or both of 1,3-dihydroxypropane-2-oxide and 2,3-dihydroxypropane-1-oxide at a molar ratio of at least 100:1 and no greater than 10,000,000:1 (glycerol:1,3-dihydroxypropane-2-oxide and 2,3-dihydroxypropane-1-oxide). In some specific embodiments, the liquid phase comprises glycerol and one or both of 1,3-dihydroxypropane-2-oxide and 2,3-dihydroxypropane-1-oxide at a molar ratio of at least 100:1 and no greater than 100,000:1.

In some embodiments, the liquid phase comprises butane-1,2,3,4-tetrol and one or both of 1,3,4-trihydroxybutane-2-oxide and 2,3,4-trihydroxybutane-1-oxide at a molar ratio of at least 100:1 and no greater than 10,000,000:1 (butane-1,2,3,4-tetrol:1,3,4-trihydroxybutane-2-oxide and 2,3,4-trihydroxybutane-1-oxide). In some specific embodiments, the liquid phase comprises butane-1,2,3,4-tetrol and one or both of 1,3,4-trihydroxybutane-2-oxide and 2,3,4-trihydroxybutane-1-oxide at a molar ratio of at least 100:1 and no greater than 100,000:1. In some embodiments, the butane-1,2,3,4-tetrol is erythritol or threitol.

In some embodiments, the liquid phase comprises pentane-1,2,3,4,5-pentol and one, two, or each of 1,2,4,5-tetrahydroxypentane-3-oxide, 1,3,4,5-tetrahydroxypentane-2-oxide, and 2,3,4,5-tetrahydroxypentane-1-oxide at a molar ratio of at least 100:1 and no greater than 10,000,000:1 (pentane-1,2,3,4,5-pentol:1,2,4,5-tetrahydroxypentane-3-oxide, 1,3,4,5-tetrahydroxypentane-2-oxide, and 2,3,4,5-tetrahydroxypentane-1-oxide). In some specific embodiments, the liquid phase comprises pentane-1,2,3,4,5-pentol and one, two, or each of 1,2,4,5-tetrahydroxypentane-3-oxide, 1,3,4,5-tetrahydroxypentane-2-oxide, and 2,3,4,5-tetrahydroxypentane-1-oxide at a molar ratio of at least 100:1 and no greater than 100,000:1. In some embodiments, the pentane-1,2,3,4,5-pentol is arabitol, ribitol, or xylitol. In some preferred embodiments, the pentane-1,2,3,4,5-pentol is xylitol.

In some embodiments, the liquid phase comprises hexane-1,2,3,4,5,6-hexol and one, two, or each of 1,2,4,5,6-pentahydroxyhexane-3-oxide, 1,3,4,5,6-pentahydroxyhexane-2-oxide, and 2,3,4,5,6-pentahydroxyhexane-1-oxide at a molar ratio of at least 100:1 and no greater than 10,000,000:1 (hexane-1,2,3,4,5,6-hexol:1,2,4,5,6-pentahydroxyhexane-3-oxide, 1,3,4,5,6-pentahydroxyhexane-2-oxide, and 2,3,4,5,6-pentahydroxyhexane-1-oxide). In some specific embodiments, the liquid phase comprises hexane-1,2,3,4,5,6-hexol and one, two, or each of 1,2,4,5,6-pentahydroxyhexane-3-oxide, 1,3,4,5,6-pentahydroxyhexane-2-oxide, and 2,3,4,5,6-pentahydroxyhexane-1-oxide at a molar ratio of at least 100:1 and no greater than 100,000:1. In some embodiments, the hexane-1,2,3,4,5,6-hexol is fucitol, galactitol, iditol, mannitol, or sorbitol.

In some embodiments, the solvent is ethanol.

In some embodiments, the solvent is propylene glycol.

In some embodiments, the solvent is glycerol.

In some embodiments, the liquid phase has a surface-area-to-volume ratio of at least 1000 and no greater than 1,000,000,000 per meter.

In some embodiments, the composition comprises a gas phase, and the composition is an aerosol.

In some embodiments, the composition comprises a solid phase; the solid phase has a surface-area-to-volume ratio of at least 1000 and no greater than 100,000,000 per meter; the solid phase has an accessible surface area, which is the surface area of the solid phase that would be accessible to the liquid phase if the solid phase were fully-immersed in the liquid phase; and the liquid phase coats at least 10 percent of the accessible surface area of the solid phase. In some specific embodiments, the liquid phase coats at least 90 percent of the accessible surface area of the solid phase.

In some embodiments, the composition comprises the solid phase and the liquid phase at a mass ratio of at least 1:4 and no greater than 1,000:1.

In some embodiments, the solid phase comprises one or more chemical species at a combined concentration of at least 95 percent by mass, and each chemical species of the one or more chemical species is soluble in water at 37 degrees Celsius at a concentration of at least 100 grams per liter. In some specific embodiments, the one or more chemical species consist of one or more carbohydrates.

“Consist” refers to a closed set; one or more chemical species that consists of one or more carbohydrates cannot also comprise, for example, aspartame. A composition may nevertheless comprise a chemical species that is not included in the one or more chemical species, but any such additional chemical species is not included, for example, when calculating a combined concentration of the one or more chemical species.

In some embodiments, the one or more carbohydrates are selected from butane-1,2,3,4-tetrol, pentane-1,2,3,4,5-pentol, and hexane-1,2,3,4,5,6-hexol. In some specific embodiments, the one or more carbohydrates consist of one or more of erythritol, threitol, arabitol, ribitol, xylitol, fucitol, galactitol, iditol, mannitol, and sorbitol. In some preferred embodiments, the one or more carbohydrates comprise xylitol.

In some embodiments, each cation of the plurality of cations is selected from lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); copper (II) cation (“Cu++”); ammonium (“NH₄+”); protonated ethanolamine; choline; protonated lysine; protonated arginine; and protonated sphingosine. In some specific embodiments, the plurality of cations consist of one or both of sodium cation and potassium cation.

In some embodiments, the anion has general structure I, II, or III

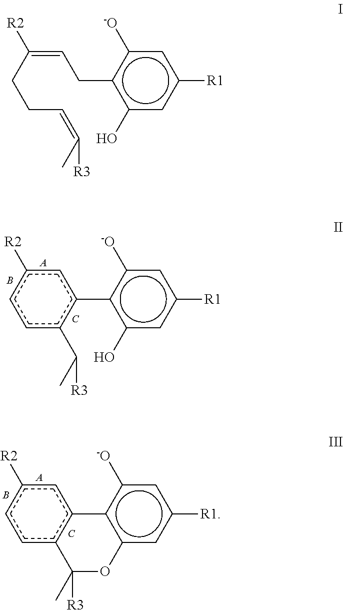

The dotted lines in general structures II and III depict the bonding pattern of either cyclohexane, phenyl, or a cyclohexene that comprises exactly one double bond, which occurs at A, B, or C.
R1 is selected from hydro; a straight or branched C1-C12 alkyl that is optionally substituted with one or more of hydroxy, cyano, phenyl, a cycloalkyl, 1, 2, or 3 halogens, and oxa; and a straight or branched C2-C12 alkenyl or alkynyl that is optionally substituted with one or more of hydroxy, cyano, phenyl, a cycloalkyl, 1, 2, or 3 halogens, and oxa.
“Straight or branched C1-C12 alkyl” refers to a straight or branched hydrocarbon chain having 1, 2, 3, 4, 5, 6, 7, 8, 9, 10, 11, or 12 carbon atoms, wherein all carbon-carbon bonds in the hydrocarbon chain are single bonds.
“Substituted with one or more of hydroxy, cyano, phenyl, a cycloalkyl, 1, 2, or 3 halogens” refers to the independent substitution of one or more hydrogen atoms of a hydrocarbon chain with hydroxy, cyano, phenyl, a cycloalkyl, or a halogen.
“Cycloalkyl” refers to cyclopropyl, cyclobutyl, cyclopentyl, cyclohexyl, cycloheptyl, and adamantyl. When a hydrocarbon chain is substituted by a cycloalkyl, then either (i) a single hydrogen atom of the hydrocarbon chain is substituted with the cycloalkyl such that the cycloalkyl does not include any carbon atom of the hydrocarbon chain, or (ii) two hydrogen atoms of the hydrocarbon chain are substituted with the cycloalkyl such that the cycloalkyl comprises one or more carbon atoms of the hydrocarbon chain. The carbon atoms of a cycloalkyl are included in the carbon atoms of a hydrocarbon chain that is substituted with the cycloalkyl when counting the number of carbon atoms in the hydrocarbon chain.
“Halogen” refers to F, Cl, Br, and I.
“Substituted with oxa” refers to the substitution of a carbon atom of a hydrocarbon chain with an oxygen atom.
“Straight or branched C2-C12 alkenyl” refers to a straight or branched hydrocarbon chain having 2, 3, 4, 5, 6, 7, 8, 9, 10, 11, or 12 carbon atoms, wherein at least one carbon-carbon bond in the hydrocarbon chain is a double bond and no carbon-carbon bond in the hydrocarbon chain is a triple bond.
“Straight or branched C2-C12 alkynyl” refers to a straight or branched hydrocarbon chain having 2, 3, 4, 5, 6, 7, 8, 9, 10, 11, or 12 carbon atoms, wherein at least one carbon-carbon bond in the hydrocarbon chain is a triple bond.
R2 is selected from hydro; hydroxy; oxo; a straight or branched C1-C5 alkyl or alkenyl that is optionally substituted with hydroxy, oxo, 1, 2, or 3 halogens, or oxa; and a straight or branched C2-C5 alkynyl that is optionally substituted with hydroxy, oxo, 1, 2, or 3 halogens, or oxa. When R2 is oxo or an ylidene, then the anion has general structure II or III, and the dotted lines in general structures II and III depict the bonding pattern of either cyclohexane or a cyclohexene that comprises exactly one double bond, which occurs at C.
“Straight or branched C1-C5 alkyl” refers to a straight or branched hydrocarbon chain having 1, 2, 3, 4, or 5 carbon atoms, wherein all carbon-carbon bonds in the hydrocarbon chain are single bonds.
“Straight or branched C1-C5 alkenyl” refers to a straight or branched hydrocarbon chain having 1, 2, 3, 4, or 5 carbon atoms, wherein at least one carbon-carbon bond in the hydrocarbon chain is a double bond and no carbon-carbon bond in the hydrocarbon chain is a triple bond. The at least one carbon-carbon double bond can optionally comprise or consist of an ylidene double bond.
“Ylidene” refers to either an R2 that includes a carbon-carbon double bond that connects the R2 to general structure II or III or an R3 that includes a carbon-carbon double bond that connects the R3 to general structure II. An example of an ylidene is methylidene.
“Straight or branched C2-C5 alkynyl” refers to a straight or branched hydrocarbon chain having 2, 3, 4, or 5 carbon atoms, wherein at least one carbon-carbon bond in the hydrocarbon chain is a triple bond.
R3 is selected from hydro; a straight or branched C1-C5 alkyl or alkenyl that is optionally substituted with hydroxy or 1, 2, or 3, halogens; and a straight or branched C2-C5 alkynyl that is optionally substituted with hydroxy or 1, 2, or 3, halogens. When R3 is an ylidene, then the anion has general structure II.

In some embodiments, the anion has the general structure I; R1 is a straight or branched C1-C12 alkyl that is optionally substituted with phenyl; R2 is methyl; and R3 is methyl.

In some embodiments, the anion has the general structure II; R1 is a straight or branched C1-C12 alkyl that is optionally substituted with phenyl or cycloalkyl; R2 is hydro, hydroxy, methyl, hydroxymethyl, oxo, formyl, acetyl, (methoxy)carbonyl, (ethoxy)carbonyl, or [(isopropyl)oxy]carbonyl; and R3 is methyl, methylidene, hydroxymethyl, or fluoromethyl.

In some embodiments, the anion has the general structure III; R1 is a straight or branched C1-C12 alkyl that is optionally substituted with phenyl or cycloalkyl; R2 is hydro, hydroxy, methyl, hydroxymethyl, oxo, formyl, acetyl, (methoxy)carbonyl, (ethoxy)carbonyl, or [(isopropyl)oxy]carbonyl; and R3 is methyl.

In some embodiments, R1 is hydro; methyl; ethyl; propyl; butyl; pentyl; hexyl; heptyl; octyl; nonyl; decyl; undecyl; dodecyl; prop-2-yl; but-2-yl; pent-2-yl; hex-2-yl; hept-2-yl; octan-2-yl; nonan-2-yl; dec-2-yl; undec-2-yl; dodec-2-yl; 2-methylpropyl; 2-methylbutyl; 2-methylpentyl; 2-methylhexyl; 2-methylheptyl; 2-methyloctyl; 2-methylnonyl; 2-methyldecyl; 2-methylundecyl; 2-methylprop-2-yl; 2-methylbut-2-yl; 2-methylpent-2-yl; 2-methylhex-2-yl; 2-methylhept-2-yl; 2-methyloct-2-yl; 2-methylnonan-2-yl; 2-methyldec-2-yl; 2-methylundec-2-yl; 3-methylbut-2-yl; 3-methylpent-2-yl; 3-methylhex-2-yl; 3-methylhept-2-yl; 3-methyloct-2-yl; 3-methylnonan-2-yl; 3-methyldec-2-yl; 3-methylundec-2-yl; 2,3-dimethylbut-2-yl; 2,3-dimethylpent-2-yl; 2,3-dimethylhex-2-yl; 2,3-dimethylhept-2-yl; 2,3-dimethyloct-2-yl; 2,3-dimethylnonan-2-yl; 2,3-dimethyldec-2-yl; cyclopropyl; 1-methylcyclopropyl; 1-ethylcyclopropyl; 1-propylcyclopropyl; 1-butylcyclopropyl; 1-pentylcyclopropyl; 1-hexylcyclopropyl; 1-heptylcyclopropyl; 1-octylcyclopropyl; 1-nonylcyclopropyl; cyclobutyl; 1-methylcyclobutyl; 1-ethylcyclobutyl; 1-propylcyclobutyl; 1-butylcyclobutyl; 1-pentylcyclobutyl; 1-hexylcyclobutyl; 1-heptylcyclobutyl; 1-octylcyclobutyl; cyclopentyl; 1-methylcyclopentyl; 1-ethylcyclopentyl; 1-propylcyclopentyl; 1-butylcyclopentyl; 1-pentylcyclopentyl; 1-hexylcyclopentyl; 1-heptylcyclopentyl; cyclohexyl; 1-methylcyclohexyl; 1-ethylcyclohexyl; 1-propylcyclohexyl; 1-butylcyclohexyl; 1-pentylcyclohexyl; 1-hexylcyclohexyl; ethenyl; prop-1-enyl; but-1-enyl; pent-1-enyl; hex-1-enyl; hept-1-enyl; oct-1-enyl; nonan-1-enyl; dec-1-enyl; undec-1-enyl; dodec-1-enyl; ethynyl; prop-1-ynyl; but-1-ynyl; pent-1-ynyl; hex-1-ynyl; hept-1-ynyl; oct-1-ynyl; nonan-1-ynyl; dec-1-ynyl; undec-1-ynyl; dodec-1-ynyl; 2-phenylethyl; 2-phenylprop-2-yl; adamant-1-yl; adamant-2-yl; 6-halohex-2-enyl; 6-halohex-2-ynyl; or 2-methyl-6-halohex-2-yl. In some specific embodiments, R1 is methyl, ethyl, propyl, butyl, pentyl, hexyl, or heptyl. In some preferred embodiments, R1 is propyl or pentyl.

In some preferred embodiments, R2 is methyl.

In some embodiments, the anion has general structure I or III, and R3 is methyl.

In some embodiments, the anion has general structure II, and R3 is methylidene.

In some very specific embodiments, the anion is 2-geranyl-3-hydroxy-5-pentylphenolate.

In some very specific embodiments, the anion is 2-geranyl-3-hydroxy-5-propylphenolate.

In some very specific embodiments, the anion is 3-hydroxy-2-(6-isopropenyl-3-methylcyclohex-2-enyl)-5-pentylphenolate.

In some very specific embodiments, the anion is 3-hydroxy-2-(6-isopropenyl-3-methylcyclohex-2-enyl)-5-propylphenolate.

In some very specific embodiments, the anion is 6,6,9-trimethyl-3-pentyl-6a,7,8,10a-tetrahydro-6H-benzo[c]chromene-1-oxide.

In some very specific embodiments, the anion is 6,6,9-trimethyl-3-propyl-6a,7,8,10a-tetrahydro-6H-benzo[c]chromene-1-oxide.

In some very specific embodiments, the anion is 6,6,9-trimethyl-3-pentyl-6H-benzo[c]chromene-1-oxide.

In some very specific embodiments, the anion is 6,6,9-trimethyl-3-propyl-6H-benzo[c]chromene-1-oxide.

In some embodiments, the combined equivalent concentration of the plurality of cations is at least micromolar and no greater than 500 millimolar; and the combined molar concentration of the plurality of anions is at least 5 micromolar and no greater than 500 millimolar.

In some embodiments, the composition comprises an oral Tmax of less than 120 minutes, wherein the oral Tmax is the time it takes to achieve a maximum blood concentration of the molecule in a human following oral administration of the composition. In some specific embodiments, the composition comprises an oral Tmax of less than 30 minutes.

In some embodiments, the composition comprises an oral bioavailability of at least 10 percent, wherein the composition comprises a combined amount of the anion and the molecule; and the oral bioavailability is an amount of the molecule that would be expected to enter the blood of a human following oral administration of the composition to the human divided by the combined amount. In some specific embodiments, the composition comprises an oral bioavailability of at least 20 percent.

In some embodiments, the composition is for use as a medicament.

In some embodiments, the composition is formulated to convert the anion into the cannabinoid molecule ex vivo prior to administering the composition to a subject.

In some embodiments, the composition is formulated to convert the anion into the cannabinoid molecule in situ subsequent to administering the composition to a subject.

In some embodiments, the composition is formulated for enteral; oral; rectal; sublingual; sublabial; buccal; intranasal; inhalational; transmucosal; topical; transdermal; intravenous; intramuscular; subcutaneous; intradermal; intraocular; parenteral; intrathecal; intralesional; or intratumoral administration. In some specific embodiments, the composition is formulated for topical or transdermal administration.

In some embodiments, the composition is a liquid; beverage; elixir; tincture; syrup; pill; tablet; capsule; gel cap; soft gel; gummy; gel; cream; lotion; balm; salve; ointment; dermal patch; transdermal patch; powder; or suppository. In some specific embodiments, the composition is a gel; cream; lotion; balm; salve; ointment; dermal patch; or transdermal patch.

In some embodiments, the composition is formulated for oral administration to a subject; the composition is formulated to allow the conversion of the anion into the cannabinoid molecule before the anion reaches the stomach of the subject; and the composition is formulated to allow absorption of the cannabinoid molecule by the epithelial lining of the gastrointestinal tract between the lips and the stomach, excluding the stomach and the outer surfaces of the lips, and including the esophagus and the inner surfaces of the lips.

Various aspects of the disclosure relate to a method to administer a cannabinoid, comprising providing a composition described anywhere in the disclosure, and administering the composition to a subject.

In some embodiments, the method comprises converting the anion into the cannabinoid molecule ex vivo prior to the administering.

In some embodiments, the method comprises converting the anion into the cannabinoid molecule in situ subsequent to the administering.

In some embodiments, the subject is human.

In some embodiments, the subject presents with a health condition or a risk of developing a health condition. In some specific embodiments, the composition is administered in an amount that is effective to treat or prophylactically prevent the health condition.

“Treat” refers to at least one of: to cure a health condition; to increase the probability that a health condition will be cured; to shorten the time over which a health condition is cured; to increase the probability that the time necessary to cure a health condition will be shortened; to decrease the severity of a health condition; to increase the probability that the severity of a health condition will decrease; to shorten the time over which the severity of a health condition is decreased; to increase the probability that the time necessary to decrease the severity of a health condition will be shortened; to inhibit a health condition from worsening; to increase the probability that a health condition will not worsen; to delay the worsening of a health condition; to increase the probability that the worsening of a health condition will be delayed; to inhibit the occurrence or recurrence of a health condition; to decrease the probability that a health condition will occur or reoccur; to delay the onset of a health condition; to increase the probability that the onset of a health condition will be delayed; to alleviate at least one symptom of a health condition; to increase the probability that at least one symptom of a health condition will be alleviated; to shorten the time over which at least one symptom of a health condition is alleviated; to increase the probability that the time necessary to alleviate at least one symptom of a health condition will be shortened; to decrease the severity of at least one symptom of a health condition; to increase the probability that the severity of at least one symptom of a health condition will be decreased; to shorten the time over which the severity of at least one symptom of a health condition is decreased; to increase the probability that the time necessary to decrease the severity of at least one symptom of a health condition will be shortened; to inhibit at least one symptom of a health condition from worsening; to increase the probability that at least one symptom of a health condition will not worsen; to delay the worsening of at least one symptom of a health condition; to increase the probability that the worsening of at least one symptom of a health condition will be delayed; to inhibit at least one symptom of a health condition from occurring or reoccurring; to decrease the probability that at least one symptom of a health condition will occur or reoccur; to delay the onset of at least one symptom of a health condition; and to increase the probability that the onset of at least one symptom of a health condition will be delayed.

In some embodiments, the amount comprises a combined amount of the anion and the molecule, which is at least 0.02 and no greater than 4 milligrams per kilogram bodyweight of the subject per day.

In some embodiments, the amount comprises a combined amount of the anion and the molecule, which is at least 1 and no greater than 200 milligrams per day.

In some embodiments, the amount comprises a combined amount of the anion and the molecule, which is at least 1 and no greater than 200 milligrams.

In some embodiments, the molecule is effective to treat or prophylactically prevent the health condition; the amount lacks the molecule at a concentration that is effective to treat or prophylactically prevent the health condition; and the amount comprises the anion at a concentration that is effective to treat or prophylactically prevent the health condition.

In some embodiments, the health condition is type 2 diabetes mellitus.

In some embodiments, the health condition is metabolic syndrome.

In some embodiments, the health condition is hypertension.

In some embodiments, the health condition is pre-hypertension.

In some embodiments, the health condition is a neurodegenerative disease or neuropathy.

In some embodiments, the health condition is peripheral neuropathy.

In some embodiments, the health condition is mild cognitive impairment, Alzheimer's Disease, Parkinson's Disease, amyotrophic lateral sclerosis (“ALS”), or Huntington's disease.

In some embodiments, the health condition is an autoimmune disorder.

In some embodiments, the health condition is arthritis, ankylosing spondylitis, an inflammatory autoimmune-mediated arthritis, rheumatoid arthritis, psoriatic arthritis, psoriasis, plaque psoriasis, lupus, Sjogren's syndrome, inflammatory bowel disease, Crohn's disease, or ulcerative colitis.

In some embodiments, the health condition is arthritis; inflammatory autoimmune-mediated arthritis; rheumatoid arthritis; juvenile idiopathic arthritis; polyarticular juvenile idiopathic arthritis; osteoarthritis; enthesitis-related arthritis; psoriatic arthritis; psoriasis; plaque psoriasis; hidradenitis suppurativa; sarcoidosis; pulmonary sarcoidosis; bone sarcoidosis; lupus; axial spondyloarthritis; ankylosing spondylitis; Dupuytren's disease; uveitis; non-infectious uveitis; adhesive capsulitis; Sjogren's syndrome; inflammatory bowel disease; Crohn's disease; ulcerative colitis; or smoking-cessation-induced ulcerative colitis.

In some embodiments, the health condition is arthritis.

In some embodiments, the health condition is inflammatory autoimmune-mediated arthritis.

In some embodiments, the health condition is rheumatoid arthritis.

In some embodiments, the health condition is juvenile idiopathic arthritis.

In some embodiments, the health condition is polyarticular juvenile idiopathic arthritis.

In some embodiments, the health condition is osteoarthritis.

In some embodiments, the health condition is enthesitis-related arthritis.

In some embodiments, the health condition is psoriatic arthritis.

In some embodiments, the health condition is psoriasis.

In some embodiments, the health condition is plaque psoriasis.

In some embodiments, the health condition is hidradenitis suppurativa.

In some embodiments, the health condition is sarcoidosis.

In some embodiments, the health condition is pulmonary sarcoidosis.

In some embodiments, the health condition is bone sarcoidosis.

In some embodiments, the health condition is lupus.

In some embodiments, the health condition is axial spondyloarthritis.

In some embodiments, the health condition is ankylosing spondylitis.

In some embodiments, the health condition is Dupuytren's disease.

In some embodiments, the health condition is uveitis.

In some embodiments, the health condition is non-infectious uveitis.

In some embodiments, the health condition is adhesive capsulitis.

In some embodiments, the health condition is Sjogren's syndrome.

In some embodiments, the health condition is inflammatory bowel disease.

In some embodiments, the health condition is Crohn's disease.

In some embodiments, the health condition is ulcerative colitis.

In some embodiments, the health condition is smoking-cessation-induced ulcerative colitis.

In some embodiments, the health condition is a headache, a migraine headache, or an episodic migraine.

In some embodiments, the health condition is pain. In some specific embodiments, the health condition is nociceptive pain; allodynia; chronic pain; intractable pain; back pain; lower back pain; chronic back pain; sciatica; spinal stenosis; chronic radiculopathy; post laminectomy syndrome; stomach pain; visceral pain; interstitial cystitis; postherpetic neuralgia; sickle cell anemia; sickle cell disease; cancer pain; intractable cancer pain; fibromyalgia; neurogenic pain; neuropathic pain; peripheral neuropathy; inflammatory demyelinating polyneuropathy; peripheral pain; reflex sympathetic dystrophy; residual limb pain; idiopathic pain; psychogenic pain; causalgia; complex regional pain syndrome; complex regional pain syndrome type I; complex regional pain syndrome type II; gout; epidermolysis bullosa; hemorrhoids; or constipation.

In some embodiments, the health condition is elevated intraocular pressure or glaucoma.

In some embodiments, the health condition is liver disease; non-alcoholic fatty liver disease (“NAFLD”); non-alcoholic steatohepatitis (“NASH”); liver cirrhosis; decompensated cirrhosis; hepatic encephalopathy; hepatitis; or hepatitis C.

In some embodiments, the health condition is nausea; cachexia; anorexia; bulimia; vomiting; motion sickness; cancer chemotherapy-induced anorexia; human deficiency virus (“HIV”) infection related nausea or cachexia; or acquired immune deficiency syndrome (“AIDS”) related nausea or cachexia.

In some embodiments, the health condition is anxiety; generalized anxiety disorder; a specific phobia; agoraphobia; social anxiety disorder; separation anxiety disorder; panic disorder; selective mutism; obsessive—compulsive disorder; depression; a major depressive disorder with psychotic feature(s); psychotic depression; paranoia; psychosis; early psychosis; an unspecified psychosis; an unspecified reactive psychosis; a psychotic disorder; a brief psychotic disorder; a debilitating psychiatric disorder; schizophrenia; schizophreniform disorder; schizoaffective disorder; paranoid personality disorder; schizoid personality disorder; schizotypal personality disorder; a shared psychotic disorder; a shared paranoia disorder; a delusional disorder; bipolar disorder; bipolar I disorder; bipolar II disorder; mania; manic disorder; or manic-depressive psychosis.

In some embodiments, the health condition is an addiction. In some specific embodiments, the health condition is an addiction to alcohol, tobacco, nicotine, an opiate, a stimulant, a prescription drug, gambling, food, shopping, the internet, or sex.

In some embodiments, the health condition is drug withdrawal; alcohol withdrawal syndrome; nicotine withdrawal; opioid withdrawal; cannabis withdrawal; benzodiazepine withdrawal syndrome; antidepressant discontinuation syndrome; antipsychotic withdrawal syndrome; addictive behavior; or cannabis use disorder.

In some embodiments, the health condition is attention deficit hyperactivity disorder (“ADHD”); autism or an autism spectrum disorder; Asperger syndrome; fragile X syndrome; a pervasive developmental disorder not otherwise specified (“PDD-NOS”); a childhood disintegrative disorder; or Tourette's syndrome.

In some embodiments, the health condition is Down syndrome.

In some embodiments, the health condition is post-traumatic stress disorder (“PTSD”).

In some embodiments, the health condition is asthma; respiratory disease; chronic lower respiratory disease; or chronic obstructive pulmonary disease (“COPD”).

In some embodiments, the health condition is insomnia; sleep apnea; obstructive sleep apnea; or restless legs syndrome.

In some embodiments, the health condition is cramping; muscle spasms; spasticity; spasmodic torticollis; a dyskinetic movement disorder; dystonia; intractable spasticity; intractable skeletal muscular spasticity; inclusion body myositis; myasthenia gravis; muscular dystrophy; Duchenne muscular dystrophy; muscle tremor; cerebellar tremor; dystonic tremor; essential tremor; orthostatic tremor; Parkinsonian tremor; physiological tremor; psychogenic tremor; rubral tremor; or nystagmus.

In some embodiments, the health condition is a seizure disorder. In some specific embodiments, the health condition is recurrent focal seizures; recurrent generalized seizures; recurrent absence seizures; recurrent myoclonic-absence seizures; recurrent myoclonus; recurrent myoclonic seizures; recurrent tonic seizures; recurrent tonic-clonic seizures; recurrent atonic seizures; recurrent chronic seizures; epilepsy; recurrent epileptic spasms; refractory epilepsy; intractable epilepsy; treatment-resistant epilepsy; Lennox-Gastaut syndrome; Dravet syndrome; febrile infection related epilepsy syndrome (“FIRES”); juvenile myoclonic epilepsy; myoclonic absence seizures (“MAS”); myoclonic astatic epilepsy (“MAE”); tuberous sclerosis complex (“TSC”); Rett syndrome; or Angelman syndrome.

In some embodiments, the health condition is a neurological condition. In some specific embodiments, the health condition is stroke; hemorrhagic stroke; ischemic stroke; neonatal hypoxic-ischemic encephalopathy (“NHIE”); hydrocephalus; hydromyelia; traumatic brain injury (“TBI”); post-concussion syndrome; chronic traumatic encephalopathy; a spinal cord injury; a spinal cord disease; syringomyelia; Tarlov cysts; cystic fibrosis; cerebral palsy; spinocerebellar ataxia; a neural-tube defect; neuropathy; a brain tumor; glioblastoma multiforme; glioblastoma astrocytoma; neurofibromatosis; Arnold-Chiari malformation; or multiple sclerosis.

In some embodiments, the health condition is a connective tissue disorder. In some specific embodiments, the health condition is Ehlers-Danlos syndrome; fibrous dysplasia; osteogenesis imperfecta; nail-patella syndrome; idiopathic pulmonary fibrosis; bone loss; bone loss caused by a bone fracture; bone loss caused by a surgical procedure; a periodontal defect; periodontal disease; osteopenia; an osteolytic bone disease; osteoporosis; age-related osteoporosis; hormone-related osteoporosis; hypogonadism-related osteoporosis; diabetes-related osteoporosis; glucocorticoid-related osteoporosis; or disuse osteoporosis.

In some embodiments, the health condition is a carcinoma; sarcoma; lymphoma; leukemia; germ cell tumor; or blastoma. In some specific embodiments, the health condition is condition is brain cancer; ovarian cancer; breast cancer; vaginal cancer; vulvar cancer; uterine cancer; cervical cancer; endometrial cancer; prostate cancer; testicular cancer; penile cancer; liver cancer; intrahepatic bile duct cancer; lung cancer; small cell lung cancer; non-small cell lung cancer; bronchial cancer; mesothelioma; pancreatic cancer; gall bladder cancer; non-melanoma skin cancer; melanoma; Kaposi sarcoma; thyroid cancer; head and neck cancer; nasopharyngeal cancer; oropharyngeal cancer; hypopharyngeal cancer; laryngeal cancer; oral cavity cancer; tongue cancer; mouth cancer; salivary gland cancer; esophageal cancer; gastric cancer; colorectal cancer; colon cancer; rectal cancer; anal cancer; kidney cancer; renal cell cancer; renal pelvis cancer; bladder cancer; urethral cancer; Hodgkin lymphoma; non-Hodgkin's lymphoma; myeloma; multiple myeloma; acute lymphocytic leukemia; chronic lymphocytic leukemia; acute myeloid leukemia; chronic myeloid leukemia; osteosarcoma; or soft tissue cancer.

In some embodiments, aberrant TNF-alpha signaling either causes the health condition; and the pharmaceutical composition is administered to inhibit TNF-alpha-mediated signaling pathways.

In some embodiments, aberrant TNF-alpha signaling exacerbates the health condition; and the pharmaceutical composition is administered to inhibit TNF-alpha-mediated signaling pathways.

In some embodiments, the administering is performed by spraying the composition into the mouth of the subject.

### EXEMPLIFICATION

The examples set forth specific embodiments that do not limit the scope of the disclosure or any patent claim that matures from this document.

### Example 1. Cannabigerol and Cannabigerovarin Inhibit Pain, Inflammation, and TNF-Alpha-Mediated Signaling Pathways in a Mouse Arthritis Model

Female Sabra mice are administered either vehicle or a cannabinoid. The cannabinoid is selected from 2-geranyl-5-pentylbenzene-1,3-diol (“cannabigerol”) and 2-geranyl-5-propylbenzene-1,3-diol (“cannabigerovarin”). The cannabinoid is administered at an amount of either 1, 2, 4, 8, 16, 32, or 64 milligrams cannabinoid per kilogram mouse body weight per day by oral gavage beginning when they are two months old.

Following one month of administration of either vehicle or a cannabinoid, 30 microliters of a 2 percent weight-by-volume suspension of zymosan A in saline is injected into each right hind paw of the mice. Inflammation is measured with calipers to determine right hind paw swelling at 1, 2, 6, 12, and 24 hours following the zymosan A injection relative to left hind paw. Pain is measured with a Von Frey hair aesthesiometer to determine right hind paw sensitivity to force relative to left hind paw. Serum TNF-alpha concentration is measured at 24 hours by enzyme-linked immunosorbent assay.

Each of the cannabigerol and cannabigerovarin groups display a dose-dependent reduction in right hind paw inflammation and pain relative to vehicle control groups. Each of the cannabigerol and cannabigerovarin groups display a dose-dependent reduction in serum TNF-alpha concentration relative to vehicle control groups. These experiments suggest that each of cannabigerol and cannabigerovarin are effective at treating pain and inflammation. These experiments also suggest that each of cannabigerol and cannabigerovarin can generally inhibit TNF-alpha-mediated signaling pathways. These experiments also suggest that each of cannabigerol and cannabigerovarin will be effective in humans at treating each of pain; inflammation; arthritis; inflammatory autoimmune-mediated arthritis; rheumatoid arthritis; juvenile idiopathic arthritis; polyarticular juvenile idiopathic arthritis; osteoarthritis; enthesitis-related arthritis; psoriatic arthritis; psoriasis; plaque psoriasis; hidradenitis suppurativa; sarcoidosis; pulmonary sarcoidosis; bone sarcoidosis; lupus; axial spondyloarthritis; ankylosing spondylitis; Dupuytren's disease; uveitis; non-infectious uveitis; adhesive capsulitis; Sjogren's syndrome; inflammatory bowel disease; Crohn's disease; ulcerative colitis; and smoking-cessation-induced ulcerative colitis.

### Example 2. Cannabigerol and Cannabigerovarin Inhibit Neutrophil-Mediated Inflammation in a Mouse Arthritis Model

Male C57BL/6J mice are administered either vehicle or a cannabinoid. The cannabinoid is selected from cannabigerol and cannabigerovarin. The cannabinoid is administered at an amount of either 1, 2, 4, 8, 16, 32, or 64 milligrams cannabinoid per kilogram mouse body weight per day by oral gavage beginning when they are two months old.

Following one month of administration, mice are anesthetized with isoflurane and then 180 micrograms of zymosan A in saline is injected into the right knee of each mouse. Equivalent volumes of saline are injected into the left knee of each mouse. Mice are then injected with Neutrophil Elastase 680 FAST imaging probe (Perkin Elmer, Massachusetts, United States) and imaged 4 hours post-injection using an IVIS Spectrum In Vivo Imaging System (Perkin Elmer) to measure neutrophil-mediated inflammation. Mouse RNA is extracted and purified, and then relative adamts4 transcript, relative neutrophil elastase transcript, and relative myeloperoxidase transcript concentrations are measured following reverse transcription using real-time polymerase chain reactions.

Each of the cannabigerol and cannabigerovarin groups display a dose-dependent reduction in imaged right knee neutrophil elastase relative to vehicle control group. Each of the cannabigerol and cannabigerovarin groups display a dose-dependent reduction in adamts4 transcripts, neutrophil elastase transcripts, and myeloperoxidase transcripts relative to vehicle control groups. These experiments suggest that each of cannabigerol and cannabigerovarin inhibit neutrophil-mediated inflammation. TNF-alpha is known to increase neutrophil-mediated inflammation, which corroborates the hypothesis that each of cannabigerol and cannabigerovarin inhibit TNF-alpha-mediated signaling pathways, and which therefore also corroborates the hypothesis that each of cannabigerol and cannabigerovarin will be effective in humans at treating each of pain; inflammation; arthritis; inflammatory autoimmune-mediated arthritis; rheumatoid arthritis; juvenile idiopathic arthritis; polyarticular juvenile idiopathic arthritis; osteoarthritis; enthesitis-related arthritis; psoriatic arthritis; psoriasis; plaque psoriasis; hidradenitis suppurativa; sarcoidosis; pulmonary sarcoidosis; bone sarcoidosis; lupus; axial spondyloarthritis; ankylosing spondylitis; Dupuytren's disease; uveitis; non-infectious uveitis; adhesive capsulitis; Sjogren's syndrome; inflammatory bowel disease; Crohn's disease; ulcerative colitis; and smoking-cessation-induced ulcerative colitis.

### Example 3. Cannabigerol and Cannabigerovarin Inhibit Obesity and Liver Injury Biomarkers in Mouse Diet-Induced Obesity Models

Mice are fed either a control standard diet or a high-fat diet beginning when they are two months old. The mice are concomitantly administered either vehicle or a cannabinoid selected from cannabigerol and cannabigerovarin at an amount of either 1, 2, 4, 8, 16, 32, or 64 milligrams cannabinoid per kilogram mouse body weight per day by oral gavage.

Mouse body weights are measured daily. The mice are sacrificed when they are four months old following two months of the diet, cannabinoid (or vehicle), and weighing. Serum alanine aminotransferase (“ALT”) and serum aspartate aminotransferase (“AST”) are measured using a Reflotran Plus blood chemistry system (Roche, Switzerland). Mouse livers are fixed and then stained with hematoxylin and eosin to visualize liver steatosis.

Each of the cannabigerol and cannabigerovarin groups display a dose-dependent reduction in body weight. Each of the cannabigerol and cannabigerovarin groups display a dose-dependent reduction in serum ALT and serum AST. Each of the cannabigerol and cannabigerovarin groups display a dose-dependent reduction in liver steatosis. These experiments suggest that each of cannabigerol and cannabigerovarin can treat both obesity and liver steatosis.

### Example 4. Treatment of Cancer Pain

An individual who presented with cancer pain caused by throat cancer was administered water containing the cannabidiol anion (2-[6-isopropenyl-3-methylcyclohex-2-enyl]-3-hydroxy-5-pentylphenolate). The individual reported notable alleviation of throat pain within minutes of ingestion.

### Example 5. Treatment of Acute Back Pain

An individual who presented with back pain associated with driving for prolonged periods of time was administered a glycerol tincture containing the cannabidiol anion. The individual reported notable alleviation of the back pain within minutes of ingestion.

### Example 6. Treatment of Chronic Back Pain

An individual who presented with chronic back pain associated with standing was administered a glycerol tincture containing the cannabidiol anion. The individual reported notable alleviation of the back pain within minutes of ingestion.

### Example 7. Treatment of Chronic Hip Pain

An individual who presented with hip pain associated with sitting was administered a glycerol tincture containing the cannabidiol anion. The individual reported notable alleviation of the hip pain within minutes of ingestion.

### Example 8. Treatment of Post Laminectomy Syndrome

An 87-year-old individual with hip pain and lower back pain associated with spinal fusion surgery was administered a glycerol tincture containing the cannabidiol anion. The individual reported notable alleviation of hip pain and lower back pain within minutes of ingestion.

### Example 9. Treatment of Knee Pain

An individual who presented with knee pain associated with inflammation caused by trauma from a fall was administered a glycerol tincture containing the cannabidiol anion. The individual reported improved mobility and alleviation of knee pain within minutes of ingestion.

### Example 10. Treatment of Arthritis

An individual who was unable to fill her hydroxychloroquine prescription to treat the arthritis during the COVID-19 pandemic was administered a glycerol tincture containing the cannabidiol anion. The individual reported that the glycerol tincture containing the cannabidiol anion was more effective at treating her arthritis than hydroxychloroquine.

### Example 11. Treatment of Chronic Pain

An individual who presented with chronic pain was administered a glycerol tincture containing the cannabidiol anion. The individual reported alleviation of pain that significantly improved her standard of living.

### Example 12. Treatment of Anxiety

An individual was administered a beverage containing the cannabidiol anion prior to driving to an airport to catch a flight that was scheduled to leave within thirty minutes of the arrival at the airport. The individual reported that he would ordinarily experience anxiety during such a drive when confronted with a similar timeline, and the individual reported a notable absence of anxiety.

An individual who suffers from chronic anxiety was administered a glycerol tincture containing the cannabigerol anion. The individual reported a significant improvement in his anxiety symptoms.

### Example 13. Treatment of Social Anxiety

An individual who presented with social anxiety associated with presenting at business meetings was administered a beverage containing the cannabidiol anion before presenting. The individual reported reduced anxiety and improved performance during the business meeting.

### Example 14. Treatment of Cramping and Restless Legs Syndrome

An individual who presented with cramping and restless legs syndrome was administered a glycerol tincture containing the cannabidiol anion before sleep. The individual reported a significant improvement in sleep quality. The individual noted similar improvements after taking a glycerol tincture containing the cannabigerol anion (2-geranyl-3-hydroxy-5-pentylphenolate) before sleep.

### Example 15. Treatment of Epilepsy

A dog that suffers from chronic seizures was administered a glycerol tincture containing the cannabidiol anion immediately after the onset of a seizure. The owners of the dog reported that the glycerol tincture containing the cannabidiol anion both instantaneously and consistently arrested multiple seizures in the dog and significantly improved the quality of life of the dog.

### Example 16. Treatment of Insomnia

An individual who presented with chronic insomnia was administered a glycerol tincture containing the cannabigerol anion before sleep. The individual reported a significant improvement in the onset and quality of his sleep.

### Example 17. Treatment of Crohn's Disease

An individual who presented with Crohn's disease was administered a glycerol tincture containing the cannabigerol anion. The individual reported a significant improvement in her symptoms of Crohn's disease, which enabled here to eat problem foods that she could not previously eat without debilitating gastrointestinal distress.

### Example 18. Treatment of Inflammatory Bowel Disease

An individual who presented with Inflammatory Bowel Disease was administered a glycerol tincture containing the cannabigerol anion. The individual reported a significant improvement in her symptoms of Inflammatory Bowel Disease, which enabled here to eat problem foods that she could not previously eat without debilitating gastrointestinal distress.

### Example 19. Treatment of Plaque Psoriasis

An individual who presented with plaque psoriasis topically administered a glycerol formulation containing the cannabigerol anion. The individual reported a significant improvement in her plaque psoriasis symptoms.

### Example 20. Treatment of Peripheral Neuropathy

An individual who presented with peripheral neuropathy topically administered a glycerol formulation containing the cannabigerol anion. The individual reported a significant improvement in his peripheral neuropathy symptoms.

### Example 21. Cannabigerol and Cannabigerovarin Likely Inhibit TNF-Alpha Signaling in Humans

Examples 1 and 2 each suggest that each of cannabigerol and cannabigerovarin can inhibit TNF-alpha-mediated signaling pathways in mice, which suggests that each of the foregoing cannabinoids can treat health conditions in humans by inhibiting TNF-alpha-mediated signaling pathways. Anti-TNF-alpha pharmaceuticals such as HUMIRA®, REMICADE®, and ENBREL® are known to be effective at treating Crohn's Disease and plaque psoriasis. Examples 17 and 19 therefore corroborate the hypothesis that each of cannabigerol and cannabigerovarin can treat health conditions in humans by inhibiting TNF-alpha-mediated signaling pathways.

In light of the foregoing, the examples suggest that cannabigerol and cannabigerovarin can treat a range of health conditions in humans including pain; inflammation; arthritis; inflammatory autoimmune-mediated arthritis; rheumatoid arthritis; juvenile idiopathic arthritis; polyarticular juvenile idiopathic arthritis; osteoarthritis; enthesitis-related arthritis; psoriatic arthritis; psoriasis; plaque psoriasis; hidradenitis suppurativa; sarcoidosis; pulmonary sarcoidosis; bone sarcoidosis; lupus; axial spondyloarthritis; ankylosing spondylitis; Dupuytren's disease; uveitis; non-infectious uveitis; adhesive capsulitis; Sjogren's syndrome; inflammatory bowel disease; Crohn's disease; ulcerative colitis; and smoking-cessation-induced ulcerative colitis.

What is claimed is:

1. A composition, comprising a liquid phase that comprises a plurality of cations, an anion, a molecule, a base, and a solvent, wherein the plurality of cations, the anion, the molecule, and the base are solutes that are dissolved in the solvent; each cation of the plurality of cations is a metal cation or an ammonium cation; the molecule is a cannabinoid; the solvent is not water; the solvent has a solubility in water at 37 degrees Celsius of at least 25 grams per liter; each cation of the plurality of cations has a net charge of 1, 2, or 3; the anion has a net charge of −1; the molecule lacks a net charge; the base has a net charge of −1; the composition has a molar concentration for the anion, the molecule, the base, and each cation of the plurality of cations; the composition has an equivalent concentration for each cation of the plurality of cations, which is equal to the molar concentration of a cation multiplied by the net charge of the same cation; the composition has a combined equivalent concentration of the plurality of cations, which is equal to the sum of the equivalent concentrations of the cations of the plurality of cations; the combined equivalent concentration is no less than the molar concentration of the anion; the combined equivalent concentration is greater than the molar concentration of the base; the molar concentration of the anion is greater than the molar concentration of the molecule; the molar concentration of the solvent is at least 100 times greater than the molar concentration of the base; the liquid phase comprises the solvent at a greater concentration by mass than any other chemical species that is present in the liquid phase; the molecule has an acid dissociation constant in water of at least 50 femtomolar and no greater than 50 nanomolar for conversion of the molecule into the anion; the solvent has an acid dissociation constant in water of at least 50 attomolar for conversion of the solvent into the base; the acid dissociation constant of the molecule is at least 10 times greater than the acid dissociation constant of the solvent; the molecule has an octanol-water partition coefficient at 37 degrees Celsius that is greater than 1; the molecule has a solubility in water, which is the maximum thermodynamically-stable molar concentration of the molecule that can be dissolved in distilled water as a solute that is solvated by water at atmospheric pressure and 21 degrees Celsius; and the molar concentration of the anion is greater than the solubility of the molecule in water.

2. The composition of claim 1, wherein the liquid phase comprises water and hydroxide at a molar ratio of at least 100:1 and no greater than 10,000,000:1.

3. The composition of claim 1 or 2, wherein the liquid phase comprises water and hydroxide at a molar ratio of at least 100:1 and no greater than 100,000:1.

4. The composition of any one of claims 1-3, wherein the liquid phase comprises ethanol and ethoxide at a molar ratio of at least 100:1 and no greater than 10,000,000:1.

5. The composition of any one of claims 1-4, wherein the liquid phase comprises ethanol and ethoxide at a molar ratio of at least 100:1 and no greater than 100,000:1.

6. The composition of any one of claims 1-5, wherein the liquid phase comprises propylene glycol and one or both of 2-hydroxypropane-1-oxide and 1-hydroxypropane-2-oxide at a molar ratio of at least 100:1 and no greater than 10,000,000:1 (propylene glycol:2-hydroxypropane-1-oxide and 1-hydroxypropane-2-oxide).

7. The composition of claim 6, wherein the liquid phase comprises propylene glycol and one or both of 2-hydroxypropane-1-oxide and 1-hydroxypropane-2-oxide at a molar ratio of at least 100:1 and no greater than 100,000:1.

8. The composition of any one of claims 1-7, wherein the liquid phase comprises glycerol and one or both of 1,3-dihydroxypropane-2-oxide and 2,3-dihydroxypropane-1-oxide at a molar ratio of at least 100:1 and no greater than 10,000,000:1 (glycerol:1,3-dihydroxypropane-2-oxide and 2,3-dihydroxypropane-1-oxide).

9. The composition of claim 8, wherein the liquid phase comprises glycerol and one or both of 1,3-dihydroxypropane-2-oxide and 2,3-dihydroxypropane-1-oxide at a molar ratio of at least 100:1 and no greater than 100,000:1.

10. The composition of any one of claims 1-9, wherein the liquid phase comprises butane-1,2,3,4-tetrol and one or both of 1,3,4-trihydroxybutane-2-oxide and 2,3,4-trihydroxybutane-1-oxide at a molar ratio of at least 100:1 and no greater than 10,000,000:1 (butane-1,2,3,4-tetrol:1,3,4-trihydroxybutane-2-oxide and 2,3,4-trihydroxybutane-1-oxide).

11. The composition of claim 9, wherein the liquid phase comprises butane-1,2,3,4-tetrol and one or both of 1,3,4-trihydroxybutane-2-oxide and 2,3,4-trihydroxybutane-1-oxide at a molar ratio of at least 100:1 and no greater than 100,000:1.

12. The composition of claim 10 or 11, wherein the butane-1,2,3,4-tetrol is erythritol or threitol.

13. The composition of any one of claims 1-12, wherein the liquid phase comprises pentane-1,2,3,4,5-pentol and one, two, or each of 1,2,4,5-tetrahydroxypentane-3-oxide, 1,3,4,5-tetrahydroxypentane-2-oxide, and 2,3,4,5-tetrahydroxypentane-1-oxide at a molar ratio of at least 100:1 and no greater than 10,000,000:1 (pentane-1,2,3,4,5-pentol:1,2,4,5-tetrahydroxypentane-3-oxide, 1,3,4,5-tetrahydroxypentane-2-oxide, and 2,3,4,5-tetrahydroxypentane-1-oxide).

14. The composition of claim 13, wherein the liquid phase comprises pentane-1,2,3,4,5-pentol and one, two, or each of 1,2,4,5-tetrahydroxypentane-3-oxide, 1,3,4,5-tetrahydroxypentane-2-oxide, and 2,3,4,5-tetrahydroxypentane-1-oxide at a molar ratio of at least 100:1 and no greater than 100,000:1.

15. The composition of claim 13 or 14, wherein the pentane-1,2,3,4,5-pentol is arabitol, ribitol, or xylitol.

16. The composition of any one of claims 1-15, wherein the liquid phase comprises hexane-1,2,3,4,5,6-hexol and one, two, or each of 1,2,4,5,6-pentahydroxyhexane-3-oxide, 1,3,4,5,6-pentahydroxyhexane-2-oxide, and 2,3,4,5,6-pentahydroxyhexane-1-oxide at a molar ratio of at least 100:1 and no greater than 10,000,000:1 (hexane-1,2,3,4,5,6-hexol:1,2,4,5,6-pentahydroxyhexane-3-oxide, 1,3,4,5,6-pentahydroxyhexane-2-oxide, and 2,3,4,5,6-pentahydroxyhexane-1-oxide).

17. The composition of claim 16, wherein the liquid phase comprises hexane-1,2,3,4,5,6-hexol and one, two, or each of 1,2,4,5,6-pentahydroxyhexane-3-oxide, 1,3,4,5,6-pentahydroxyhexane-2-oxide, and 2,3,4,5,6-pentahydroxyhexane-1-oxide at a molar ratio of at least 100:1 and no greater than 100,000:1.

18. The composition of claim 16 or 17, wherein the hexane-1,2,3,4,5,6-hexol is fucitol, galactitol, iditol, mannitol, or sorbitol.

19. The composition of any one of claims 1-18, wherein the solvent is ethanol.

20. The composition of any one of claims 1-18, wherein the solvent is propylene glycol.

21. The composition of any one of claims 1-18, wherein the solvent is glycerol.

22. The composition of any one of claims 1-21, wherein the liquid phase has a surface-area-to-volume ratio of at least 1000 and no greater than 1,000,000,000 per meter.

23. The composition of any one of claims 1-22, comprising a gas phase, wherein the composition is an aerosol.

24. The composition of any one of claims 1-22, comprising a solid phase, wherein the solid phase has a surface-area-to-volume ratio of at least 1000 and no greater than 100,000,000 per meter; the solid phase has an accessible surface area, which is the surface area of the solid phase that would be accessible to the liquid phase if the solid phase were fully-immersed in the liquid phase; and the liquid phase coats at least 10 percent of the accessible surface area of the solid phase.

25. The composition of claim 24, wherein the liquid phase coats at least 90 percent of the accessible surface area of the solid phase.

26. The composition of claim 24 or 25, comprising the solid phase and the liquid phase at a mass ratio of at least 1:4 and no greater than 1,000:1.

27. The composition of any one of claims 24-26, wherein the solid phase comprises one or more chemical species at a combined concentration of at least 95 percent by mass, and each chemical species of the one or more chemical species is soluble in water at 37 degrees Celsius at a concentration of at least 100 grams per liter.

28. The composition of claim 27, wherein the one or more chemical species consist of one or more carbohydrates.

29. The composition of claim 28, wherein the one or more carbohydrates are selected from butane-1,2,3,4-tetrol, pentane-1,2,3,4,5-pentol, and hexane-1,2,3,4,5,6-hexol.

30. The composition of claim 28 or 29, wherein the one or more carbohydrates consist of one or more of erythritol, threitol, arabitol, ribitol, xylitol, fucitol, galactitol, iditol, mannitol, and sorbitol.

31. The composition of any one of claims 1-30, wherein each cation of the plurality of cations is selected from lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); copper (II) cation (“Cu++”); ammonium (“NH₄+”); protonated ethanolamine; choline; protonated lysine; protonated arginine; and protonated sphingosine.

32. The composition of any one of claims 1-31, wherein the plurality of cations consist of one or both of sodium cation and potassium cation.

33. The composition of any one of claims 1-32, wherein (i) the anion has general structure I, II, or III 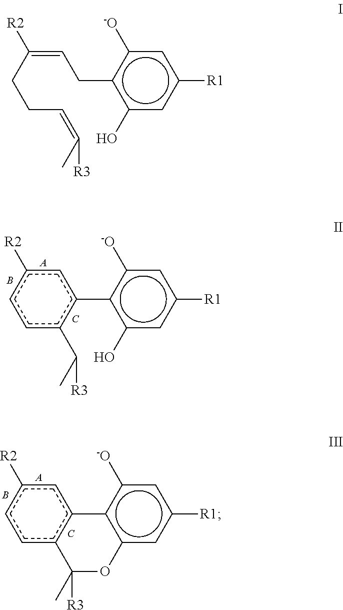
(ii) R1 is selected from hydro; a straight or branched C1-C12 alkyl that is optionally substituted with one or more of hydroxy, cyano, phenyl, a cycloalkyl, 1, 2, or 3 halogens, and oxa; and a straight or branched C2-C12 alkenyl or alkynyl that is optionally substituted with one or more of hydroxy, cyano, phenyl, a cycloalkyl, 1, 2, or 3 halogens, and oxa; (iii) R2 is selected from hydro; hydroxy; oxo; a straight or branched C1-C5 alkyl or alkenyl that is optionally substituted with hydroxy, oxo, 1, 2, or 3 halogens, or oxa; and a straight or branched C2-C5 alkynyl that is optionally substituted with hydroxy, oxo, 1, 2, or 3 halogens, or oxa; (iv) R3 is selected from hydro; a straight or branched C1-C5 alkyl or alkenyl that is optionally substituted with hydroxy or 1, 2, or 3, halogens; and a straight or branched C2-C5 alkynyl that is optionally substituted with hydroxy or 1, 2, or 3, halogens; (v) the dotted lines in general structures II and III depict the bonding pattern of either cyclohexane, phenyl, or a cyclohexene that comprises exactly one double bond, which occurs at A, B, or C; (vi) when R2 is oxo or an ylidene, then the anion has general structure II or III, and the dotted lines in general structures II and III depict the bonding pattern of either cyclohexane or a cyclohexene that comprises exactly one double bond, which occurs at C; and (vii) when R3 is an ylidene, then the anion has general structure II.

34. The composition of claim 33, wherein the anion has the general structure I; R1 is a straight or branched C1-C12 alkyl that is optionally substituted with phenyl; R2 is methyl; and R3 is methyl.

35. The composition of claim 33, wherein the anion has the general structure II; R1 is a straight or branched C1-C12 alkyl that is optionally substituted with phenyl or cycloalkyl; R2 is hydro, hydroxy, methyl, hydroxymethyl, oxo, formyl, acetyl, (methoxy)carbonyl, (ethoxy)carbonyl, or [(isopropyl)oxy]carbonyl; and R3 is methyl, methylidene, hydroxymethyl, or fluoromethyl.

36. The composition of claim 33, wherein the anion has the general structure III; R1 is a straight or branched C1-C12 alkyl that is optionally substituted with phenyl or cycloalkyl; R2 is hydro, hydroxy, methyl, hydroxymethyl, oxo, formyl, acetyl, (methoxy)carbonyl, (ethoxy)carbonyl, or [(isopropyl)oxy]carbonyl; and R3 is methyl.

37. The composition of any one of claims 33-36, wherein R1 is hydro; methyl; ethyl; propyl; butyl; pentyl; hexyl; heptyl; octyl; nonyl; decyl; undecyl; dodecyl; prop-2-yl; but-2-yl; pent-2-yl; hex-2-yl; kept-2-yl; octan-2-yl; nonan-2-yl; dec-2-yl; undec-2-yl; dodec-2-yl; 2-methylpropyl; 2-methylbutyl; 2-methylpentyl; 2-methylhexyl; 2-methylheptyl; 2-methyloctyl; 2-methylnonyl; 2-methyldecyl; 2-methylundecyl; 2-methylprop-2-yl; 2-methylbut-2-yl; 2-methylpent-2-yl; 2-methylhex-2-yl; 2-methylhept-2-yl; 2-methyloct-2-yl; 2-methylnonan-2-yl; 2-methyldec-2-yl; 2-methylundec-2-yl; 3-methylbut-2-yl; 3-methylpent-2-yl; 3-methylhex-2-yl; 3-methylhept-2-yl; 3-methyloct-2-yl; 3-methylnonan-2-yl; 3-methyldec-2-yl; 3-methylundec-2-yl; 2,3-dimethylbut-2-yl; 2,3-dimethylpent-2-yl; 2,3-dimethylhex-2-yl; 2,3-dimethylhept-2-yl; 2,3-dimethyloct-2-yl; 2,3-dimethylnonan-2-yl; 2,3-dimethyldec-2-yl; cyclopropyl; 1-methylcyclopropyl; 1-ethylcyclopropyl; 1-propylcyclopropyl; 1-butylcyclopropyl; 1-pentylcyclopropyl; 1-hexylcyclopropyl; 1-heptylcyclopropyl; 1-octylcyclopropyl; 1-nonylcyclopropyl; cyclobutyl; 1-methylcyclobutyl; 1-ethylcyclobutyl; 1-propylcyclobutyl; 1-butylcyclobutyl; 1-pentylcyclobutyl; 1-hexylcyclobutyl; 1-heptylcyclobutyl; 1-octylcyclobutyl; cyclopentyl; 1-methylcyclopentyl; 1-ethylcyclopentyl; 1-propylcyclopentyl; 1-butylcyclopentyl; 1-pentylcyclopentyl; 1-hexylcyclopentyl; 1-heptylcyclopentyl; cyclohexyl; 1-methylcyclohexyl; 1-ethylcyclohexyl; 1-propylcyclohexyl; 1-butylcyclohexyl; 1-pentylcyclohexyl; 1-hexylcyclohexyl; ethenyl; prop-1-enyl; but-1-enyl; pent-1-enyl; hex-1-enyl; hept-1-enyl; oct-1-enyl; nonan-1-enyl; dec-1-enyl; undec-1-enyl; dodec-1-enyl; ethynyl; prop-1-ynyl; but-1-ynyl; pent-1-ynyl; hex-1-ynyl; hept-1-ynyl; oct-1-ynyl; nonan-1-ynyl; dec-1-ynyl; undec-1-ynyl; dodec-1-ynyl; 2-phenylethyl; 2-phenylprop-2-yl; adamant-1-yl; adamant-2-yl; 6-halohex-2-enyl; 6-halohex-2-ynyl; or 2-methyl-6-halohex-2-yl.

38. The composition of any one of claims 33-37, wherein R1 is methyl, ethyl, propyl, butyl, pentyl, hexyl, or heptyl.

39. The composition of any one of claims 33-38, wherein R1 is propyl.

40. The composition of any one of claims 33-38, wherein R1 is pentyl.

41. The composition of any one of claims 33-40, wherein R2 is methyl.

42. The composition of any one of claims 33-41, wherein the anion has general structure I or III, and R3 is methyl.

43. The composition of any one of claims 33-41, wherein the anion has general structure II, and R3 is methylidene.

44. The composition of any one of claims 1-38 and 40-42, wherein the anion is 2-geranyl-3-hydroxy-5-pentylphenolate.

45. The composition of any one of claims 1-39, 41, and 42, wherein the anion is 2-geranyl-3-hydroxy-5-propylphenolate.

46. The composition of any one of claims 1-38, 40, 41, and 43 wherein the anion is 3-hydroxy-2-(6-isopropenyl-3-methylcyclohex-2-enyl)-5-pentylphenolate.

47. The composition of any one of claims 1-39, 41, and 43, wherein the anion is 3-hydroxy-2-(6-isopropenyl-3-methylcyclohex-2-enyl)-5-propylphenolate.

48. The composition of any one of claims 1-38 and 40-42, wherein the anion is 6,6,9-trimethyl-3-pentyl-6a,7,8,10a-tetrahydro-6H-benzo[c]chromene-1-oxide.

49. The composition of any one of claims 1-39, 41, and 42 wherein the anion is 6,6,9-trimethyl-3-propyl-6a,7,8,10a-tetrahydro-6H-benzo[c]chromene-1-oxide.

50. The composition of any one of claims 1-38 and 40-42, wherein the anion is 6,6,9-trimethyl-3-pentyl-6H-benzo[c]chromene-1-oxide.

51. The composition of any one of claims 1-39, 41, and 42 wherein the anion is 6,6,9-trimethyl-3-propyl-6H-benzo[c]chromene-1-oxide.

52. The composition of any one of claims 1-51, wherein the combined equivalent concentration of the plurality of cations is at least 5 micromolar and no greater than 500 millimolar; and the combined molar concentration of the plurality of anions is at least 5 micromolar and no greater than 500 millimolar.

53. The composition of any one of claims 1-52, comprising an oral Tmax of less than 120 minutes, wherein the oral Tmax is the time it takes to achieve a maximum blood concentration of the molecule in a human following oral administration of the composition.

54. The composition of claim 53, comprising an oral Tmax of less than 30 minutes.

55. The composition of any one of claims 1-54, comprising an oral bioavailability of at least 10 percent, wherein the composition comprises a combined amount of the anion and the molecule; and the oral bioavailability is an amount of the molecule that would be expected to enter the blood of a human following oral administration of the composition to the human divided by the combined amount.

56. The composition of claim 55, comprising an oral bioavailability of at least 20 percent.

57. The composition of any one of claims 1-56, for use as a medicament.

58. The composition of any one of claims 1-57, wherein the composition is formulated to convert the anion into the cannabinoid molecule ex vivo prior to administering the composition to a subject.

59. The composition of any one of claims 1-57, wherein the composition is formulated to convert the anion into the cannabinoid molecule in situ subsequent to administering the composition to a subject.

60. The composition of any one of claims 1-59, wherein the composition is formulated for enteral; oral; rectal; sublingual; sublabial; buccal; intranasal; inhalational; transmucosal; topical; transdermal; intravenous; intramuscular; subcutaneous; intradermal; intraocular; parenteral; intrathecal; intralesional; or intratumoral administration.

61. The composition of any one of claims 1-60, wherein the composition is a liquid; beverage; elixir; tincture; syrup; pill; tablet; capsule; gel cap; soft gel; gummy; gel; cream; lotion; balm; salve; ointment; dermal patch; transdermal patch; powder; or suppository.

62. The composition of any one of claims 1-61, wherein the composition is a gel; cream; lotion; balm; salve; ointment; dermal patch; or transdermal patch.

63. The composition of any one of claims 1-62, wherein the composition is formulated for topical or transdermal administration.

64. The composition of any one of claims 1-61, wherein the composition is formulated for oral administration to a subject; the composition is formulated to allow the conversion of the anion into the cannabinoid molecule before the anion reaches the stomach of the subject; and the composition is formulated to allow absorption of the cannabinoid molecule by the epithelial lining of the gastrointestinal tract between the lips and the stomach, excluding the stomach and the outer surfaces of the lips, and including the esophagus and the inner surfaces of the lips.

65. A method to administer a cannabinoid, comprising providing a composition according to any one of claims 1-64, and administering the composition to a subject.

66. The method of claim 65, comprising converting the anion into the cannabinoid molecule ex vivo prior to the administering.

67. The method of claim 65, comprising converting the anion into the cannabinoid molecule in situ subsequent to the administering.

68. The method of any one of claims 65-67, wherein the subject is human.

69. The method of any one of claims 65-68, wherein the subject presents with a health condition or a risk of developing a health condition.

70. The method of claim 69, wherein the composition is administered in an amount that is effective to treat or prophylactically prevent the health condition.

71. The method of claim 70, wherein the amount comprises a combined amount of the anion and the molecule, which is at least 0.02 and no greater than 4 milligrams per kilogram bodyweight of the subject per day.

72. The method of claim 70, wherein the amount comprises a combined amount of the anion and the molecule, which is at least 1 and no greater than 200 milligrams per day.

73. The method of claim 70, wherein the amount comprises a combined amount of the anion and the molecule, which is at least 1 and no greater than 200 milligrams.

74. The method of any one of claims 70-73, wherein the molecule is effective to treat or prophylactically prevent the health condition; the amount lacks the molecule at a concentration that is effective to treat or prophylactically prevent the health condition; and the amount comprises the anion at a concentration that is effective to treat or prophylactically prevent the health condition.

75. The method of any one of claims 69-74, wherein the health condition is type 2 diabetes mellitus.

76. The method of any one of claims 69-74, wherein the health condition is metabolic syndrome.

77. The method of any one of claims 69-74, wherein the health condition is hypertension.

78. The method of any one of claims 69-74, wherein the health condition is pre-hypertension.

79. The method of any one of claims 69-74, wherein the health condition is a neurodegenerative disease or neuropathy.

80. The method of any one of claims 69-74, wherein the health condition is peripheral neuropathy.

81. The method of any one of claims 69-74, wherein the health condition is mild cognitive impairment, Alzheimer's Disease, Parkinson's Disease, amyotrophic lateral sclerosis (“ALS”), or Huntington's disease.

82. The method of any one of claims 69-74, wherein the health condition is an autoimmune disorder.

83. The method of any one of claims 69-74, wherein the health condition is arthritis, ankylosing spondylitis, an inflammatory autoimmune-mediated arthritis, rheumatoid arthritis, psoriatic arthritis, psoriasis, plaque psoriasis, lupus, Sjogren's syndrome, inflammatory bowel disease, Crohn's disease, or ulcerative colitis.

84. The method of any one of claims 69-74, wherein the health condition is arthritis; inflammatory autoimmune-mediated arthritis; rheumatoid arthritis; juvenile idiopathic arthritis; polyarticular juvenile idiopathic arthritis; osteoarthritis; enthesitis-related arthritis; psoriatic arthritis; psoriasis; plaque psoriasis; hidradenitis suppurativa; sarcoidosis; pulmonary sarcoidosis; bone sarcoidosis; lupus; axial spondyloarthritis; ankylosing spondylitis; Dupuytren's disease; uveitis; non-infectious uveitis; adhesive capsulitis; Sjogren's syndrome; inflammatory bowel disease; Crohn's disease; ulcerative colitis; or smoking-cessation-induced ulcerative colitis.

85. The method of any one of claims 69-74, wherein the health condition is arthritis.

86. The method of any one of claims 69-74, wherein the health condition is inflammatory autoimmune-mediated arthritis.

87. The method of any one of claims 69-74, wherein the health condition is rheumatoid arthritis.

88. The method of any one of claims 69-74, wherein the health condition is juvenile idiopathic arthritis.

89. The method of any one of claims 69-74, wherein the health condition is polyarticular juvenile idiopathic arthritis.

90. The method of any one of claims 69-74, wherein the health condition is osteoarthritis.

91. The method of any one of claims 69-74, wherein the health condition is enthesitis-related arthritis.

92. The method of any one of claims 69-74, wherein the health condition is psoriatic arthritis.

93. The method of any one of claims 69-74, wherein the health condition is psoriasis.

94. The method of any one of claims 69-74, wherein the health condition is plaque psoriasis.

95. The method of any one of claims 69-74, wherein the health condition is hidradenitis suppurativa.

96. The method of any one of claims 69-74, wherein the health condition is sarcoidosis.

97. The method of any one of claims 69-74, wherein the health condition is pulmonary sarcoidosis.

98. The method of any one of claims 69-74, wherein the health condition is bone sarcoidosis.

99. The method of any one of claims 69-74, wherein the health condition is lupus.

100. The method of any one of claims 69-74, wherein the health condition is axial spondyloarthritis.

101. The method of any one of claims 69-74, wherein the health condition is ankylosing spondylitis.

102. The method of any one of claims 69-74, wherein the health condition is Dupuytren's disease.

103. The method of any one of claims 69-74, wherein the health condition is uveitis.

104. The method of any one of claims 69-74, wherein the health condition is non-infectious uveitis.

105. The method of any one of claims 69-74, wherein the health condition is adhesive capsulitis.

106. The method of any one of claims 69-74, wherein the health condition is Sjogren's syndrome.

107. The method of any one of claims 69-74, wherein the health condition is inflammatory bowel disease.

108. The method of any one of claims 69-74, wherein the health condition is Crohn's disease.

109. The method of any one of claims 69-74, wherein the health condition is ulcerative colitis.

110. The method of any one of claims 69-74, wherein the health condition is smoking-cessation-induced ulcerative colitis.

111. The method of any one of claims 69-74, wherein the health condition is a headache, a migraine headache, or an episodic migraine.

112. The method of any one of claims 69-74, wherein the health condition is pain.

113. The method of any one of claims 69-74, wherein the health condition is nociceptive pain; allodynia; chronic pain; intractable pain; back pain; lower back pain; chronic back pain; sciatica; spinal stenosis; chronic radiculopathy; post laminectomy syndrome; stomach pain; visceral pain; interstitial cystitis; postherpetic neuralgia; sickle cell anemia; sickle cell disease; cancer pain; intractable cancer pain; fibromyalgia; neurogenic pain; neuropathic pain; peripheral neuropathy; inflammatory demyelinating polyneuropathy; peripheral pain; reflex sympathetic dystrophy; residual limb pain; idiopathic pain; psychogenic pain; causalgia; complex regional pain syndrome; complex regional pain syndrome type I; complex regional pain syndrome type II; gout; epidermolysis bullosa; hemorrhoids; or constipation.

114. The method of any one of claims 69-74, wherein the health condition is elevated intraocular pressure or glaucoma.

115. The method of any one of claims 69-74, wherein the health condition is liver disease; non-alcoholic fatty liver disease (“NAFLD”); non-alcoholic steatohepatitis (“NASH”); liver cirrhosis; decompensated cirrhosis; hepatic encephalopathy; hepatitis; or hepatitis C.

116. The method of any one of claims 69-74, wherein the health condition is nausea; cachexia; anorexia; bulimia; vomiting; motion sickness; cancer chemotherapy-induced anorexia; human deficiency virus (“HIV”) infection related nausea or cachexia; or acquired immune deficiency syndrome (“AIDS”) related nausea or cachexia.

117. The method of any one of claims 69-74, wherein the health condition is anxiety; generalized anxiety disorder; a specific phobia; agoraphobia; social anxiety disorder; separation anxiety disorder; panic disorder; selective mutism; obsessive—compulsive disorder; depression; a major depressive disorder with psychotic feature(s); psychotic depression; paranoia; psychosis; early psychosis; an unspecified psychosis; an unspecified reactive psychosis; a psychotic disorder; a brief psychotic disorder; a debilitating psychiatric disorder; schizophrenia; schizophreniform disorder; schizoaffective disorder; paranoid personality disorder; schizoid personality disorder; schizotypal personality disorder; a shared psychotic disorder; a shared paranoia disorder; a delusional disorder; bipolar disorder; bipolar I disorder; bipolar II disorder; mania; manic disorder; or manic-depressive psychosis.

118. The method of any one of claims 69-74, wherein the health condition is an addiction.

119. The method of any one of claims 69-74, wherein the health condition is an addiction to alcohol, tobacco, nicotine, an opiate, a stimulant, a prescription drug, gambling, food, shopping, the internet, or sex.

120. The method of any one of claims 69-74, wherein the health condition is drug withdrawal; alcohol withdrawal syndrome; nicotine withdrawal; opioid withdrawal; cannabis withdrawal; benzodiazepine withdrawal syndrome; antidepressant discontinuation syndrome; antipsychotic withdrawal syndrome; addictive behavior; or cannabis use disorder.

121. The method of any one of claims 69-74, wherein the health condition is attention deficit hyperactivity disorder (“ADHD”); autism or an autism spectrum disorder; Asperger syndrome; fragile X syndrome; a pervasive developmental disorder not otherwise specified (“PDD-NOS”); a childhood disintegrative disorder; or Tourette's syndrome.

122. The method of any one of claims 69-74, wherein the health condition is Down syndrome.

123. The method of any one of claims 69-74, wherein the health condition is post-traumatic stress disorder (“PTSD”).

124. The method of any one of claims 69-74, wherein the health condition is asthma; respiratory disease; chronic lower respiratory disease; or chronic obstructive pulmonary disease (“COPD”).

125. The method of any one of claims 69-74, wherein the health condition is insomnia; sleep apnea; obstructive sleep apnea; or restless legs syndrome.

126. The method of any one of claims 69-74, wherein the health condition is cramping; muscle spasms; spasticity; spasmodic torticollis; a dyskinetic movement disorder; dystonia; intractable spasticity; intractable skeletal muscular spasticity; inclusion body myositis; myasthenia gravis; muscular dystrophy; Duchenne muscular dystrophy; muscle tremor; cerebellar tremor; dystonic tremor; essential tremor; orthostatic tremor; Parkinsonian tremor; physiological tremor; psychogenic tremor; rubral tremor; or nystagmus.

127. The method of any one of claims 69-74, wherein the health condition is a seizure disorder.

128. The method of any one of claims 69-74, wherein the health condition is recurrent focal seizures; recurrent generalized seizures; recurrent absence seizures; recurrent myoclonic-absence seizures; recurrent myoclonus; recurrent myoclonic seizures; recurrent tonic seizures; recurrent tonic-clonic seizures; recurrent atonic seizures; recurrent chronic seizures; epilepsy; recurrent epileptic spasms; refractory epilepsy; intractable epilepsy; treatment-resistant epilepsy; Lennox-Gastaut syndrome; Dravet syndrome; febrile infection related epilepsy syndrome (“FIRES”); juvenile myoclonic epilepsy; myoclonic absence seizures (“MAS”); myoclonic astatic epilepsy (“MAE”); tuberous sclerosis complex (“TSC”); Rett syndrome; or Angelman syndrome.

129. The method of any one of claims 69-74, wherein the health condition is a neurological condition.

130. The method of any one of claims 69-74, wherein the health condition is stroke; hemorrhagic stroke; ischemic stroke; neonatal hypoxic-ischemic encephalopathy (“NHIE”); hydrocephalus; hydromyelia; traumatic brain injury (“TBI”); post-concussion syndrome; chronic traumatic encephalopathy; a spinal cord injury; a spinal cord disease; syringomyelia; Tarlov cysts; cystic fibrosis; cerebral palsy; spinocerebellar ataxia; a neural-tube defect; neuropathy; a brain tumor; glioblastoma multiforme; glioblastoma astrocytoma; neurofibromatosis; Arnold-Chiari malformation; or multiple sclerosis.

131. The method of any one of claims 69-74, wherein the health condition is a connective tissue disorder.

132. The method of any one of claims 69-74, wherein the health condition is Ehlers-Danlos syndrome; fibrous dysplasia; osteogenesis imperfecta; nail-patella syndrome; idiopathic pulmonary fibrosis; bone loss; bone loss caused by a bone fracture; bone loss caused by a surgical procedure; a periodontal defect; periodontal disease; osteopenia; an osteolytic bone disease; osteoporosis; age-related osteoporosis; hormone-related osteoporosis; hypogonadism-related osteoporosis; diabetes-related osteoporosis; glucocorticoid-related osteoporosis; or disuse osteoporosis.

133. The method of any one of claims 69-74, wherein the health condition is a carcinoma; sarcoma; lymphoma; leukemia; germ cell tumor; or blastoma.

134. The method of any one of claims 69-74, wherein the health condition is condition is brain cancer; ovarian cancer; breast cancer; vaginal cancer; vulvar cancer; uterine cancer; cervical cancer; endometrial cancer; prostate cancer; testicular cancer; penile cancer; liver cancer; intrahepatic bile duct cancer; lung cancer; small cell lung cancer; non-small cell lung cancer; bronchial cancer; mesothelioma; pancreatic cancer; gall bladder cancer; non-melanoma skin cancer; melanoma; Kaposi sarcoma; thyroid cancer; head and neck cancer; nasopharyngeal cancer; oropharyngeal cancer; hypopharyngeal cancer; laryngeal cancer; oral cavity cancer; tongue cancer; mouth cancer; salivary gland cancer; esophageal cancer; gastric cancer; colorectal cancer; colon cancer; rectal cancer; anal cancer; kidney cancer; renal cell cancer; renal pelvis cancer; bladder cancer; urethral cancer; Hodgkin lymphoma; non-Hodgkin's lymphoma; myeloma; multiple myeloma; acute lymphocytic leukemia; chronic lymphocytic leukemia; acute myeloid leukemia; chronic myeloid leukemia; osteosarcoma; or soft tissue cancer.

135. The method of any one of claims 69-134, wherein aberrant tumor necrosis factor alpha (“TNF-alpha”) signaling either causes the health condition; and the pharmaceutical composition is administered to inhibit TNF-alpha-mediated signaling pathways.

136. The method of any one of claims 69-135, wherein aberrant tumor necrosis factor alpha (“TNF-alpha”) signaling exacerbates the health condition; and the pharmaceutical composition is administered to inhibit TNF-alpha-mediated signaling pathways.

137. The method of any one of claims 65-136, wherein the administering is performed by spraying the composition into the mouth of the subject.
